(19) United States
(12) Patent Application Publication
(10) Pub. No.: US 2020/0268655 A1
(43) Pub. Date: Aug. 27, 2020

(54) PLASMA AEROSOL INHALATION DEVICE AND METHOD FOR THINNING THE SPUTUM

(71) Applicants: National Tsing Hua University, Hsinchu (TW); Chang Gung University, Taoyuan City (TW); National Chiao Tung University, Hsinchu City (TW)

(72) Inventors: Chi-Shuo CHEN, Hsinchu (TW); Rong-Shing CHANG, Hsinchu (TW); Jong-Shinn WU, Hsinchu City (TW); Yun-Chien CHENG, Hsinchu City (TW); Hui-Ling LIN, Taoyuan City (TW)

(21) Appl. No.: 16/794,812

(22) Filed: Feb. 19, 2020

(30) Foreign Application Priority Data
Feb. 21, 2019 (TW) 108105712

(51) Int. Cl. A61K 9/00; A61M 15/00; A61L 2/00; A61L 2/14

(52) U.S. Cl. CPC A61K 9/0078; A61L 2/14; A61L 2/0005; A61M 15/0001

(57) ABSTRACT

The present invention provides a plasma aerosol inhalation device and a method for thinning the sputum. Various radicals generated by plasma can be inhaled in an aerosol form and delivered to the respiratory system for desired efficacy.

### CROSS REFERENCE TO RELATED APPLICATION

This application claims the benefits of the Taiwan Patent Application Serial Number 108105712, filed on Feb. 21, 2019, the subject matter of which is incorporated herein by reference.

### BACKGROUND OF THE INVENTION

### 1. Field of the Invention

The present invention relates to a plasma aerosol inhalation device and method for thinning the sputum.

### 2. Description of Related Art

Aerosol inhalation therapy is an important treatment method for respiratory-related diseases. Clinically, the aerosol inhalant was applied to moisturize the respiratory system, relieve asthma attacks, slow down the local inflammation, eliminate sputum, etc. In recent years, the population suffering respiratory diseases increases due to climate change and air pollution, therefore, the market of expectorant/mucolytics is rapidly expanded. In order to improve the delivery efficiency of expectorant/mucolytics, incorporations of expectorant/mucolytics and aerosol inhalation therapy have become a common treatment.

Abnormal accumulation of sputum in the respiratory system is highly related to various respiratory diseases. For example, respiratory diseases such as asthma, cystic fibrosis, and chronic obstructive pulmonary disease often cause accumulation of larger amount of thick sputum in the respiratory tract, even cause airway obstruction. Accumulation of thick sputum not only cause breathing difficulties due to its negative effect on the scavenging effect of cilia in the trachea but also is highly related to chronic inflammation or secondary infection. Hence, all kinds of expectorant/mucolytics were often used to reduce the viscosity of sputum so that the sputum can be discharged by autologous cilia or by external force using a conduit.

The mechanisms of discharging the sputum in the respiratory tract of conventional expectorants were moisturizing the respiratory tract, degrading the molecules (such as mucin, DNA, and actin microfilament) inside the sputum, or inducing the coughing reaction. For example, the active chemical compositions, such as Guaifenesin or N-acetylcysteine, of the existing expectorants such as Mucin EX, Robitussin AC, Robitussin DAC may degrade mucin for thinning the thick sputum, so that the sputum can be discharged from the respiratory tract smoothly. However, the conventional expectorants are disadvantageous of high cost and side effects due to its cytotoxicity or unpredictable physiological response, such as asthma attack.

Accordingly, it is desirable to provide a novel therapy to treat diseases related to the respiratory system without using any chemical component or biological enzyme that might cause side effects.

### SUMMARY OF THE INVENTION

The object of the present invention is to provide a new therapy for treating respiratory system. Various radicals, such as reactive oxygen species (ROS) and reactive nitrogen species (RNS) generated by plasma are carried by an aerosol, which can be inhaled for delivering the ROS/RNS radicals to the respiratory system. Radicals generated by plasma may trigger the healing process for desired efficacy without adding chemical drugs or biological enzymes.

To achieve the object, the present invention provides a method for delivering a plasma aerosol to a respiratory system, wherein the method comprises: performing plasma-treatment to a liquid (such as water) for providing a plasma-treated liquid (such as plasma-treated water) using a plasma generating unit; nebulizing the plasma-treated liquid into a plasma aerosol (such as plasma water aerosol) using a nebulizing module; and inhaling the plasma aerosol into respiratory system through oral and/or nose. Also, the present invention provides another method for delivering a plasma aerosol to a respiratory system, wherein the method comprises: nebulizing a liquid (such as water) into an aerosol form (such as water aerosol) using a nebulizing module; performing plasma treatment to the aerosol for providing a plasma aerosol using a plasma generating unit; and inhaling the plasma aerosol into respiratory system through oral and/or nose.

In order to deliver the plasma aerosol in to the respiratory system, the present invention further provide a plasma aerosol inhalation device, which comprises: a plasma treatment module including a treatment chamber and a plasma generating unit, wherein a liquid is accommodated in the treatment chamber and the liquid in the treatment chamber is treated by a plasma generated by the plasma generating unit so as to form a plasma-treated liquid; a nebulization module interconnecting with the plasma treatment module, wherein the plasma-treated liquid exported from the plasma treatment module is nebulized into a plasma aerosol; and an inhalation element interconnecting with the nebulization module and having at least one of the nasal inhalation part and an oral inhalation part, wherein the plasma aerosol outputted by the nebulization module is transported to a respiratory system through at least one of the nasal inhalation part and the oral inhalation part. In addition, the present invention provides another plasma aerosol inhalation device, which comprises: a nebulization module nebulizing a liquid into an aerosol; a plasma treatment module including a treatment chamber and a plasma generating unit, wherein the treatment chamber is interconnected to the nebulization module, wherein the aerosol exported from the nebulization module is accommodated in the treatment chamber and the aerosol in the treated by a plasma generated by the plasma generating unit so as to form a plasma aerosol; and an inhalation element interconnected to the plasma treatment module and having at least one of a nasal inhalation part and an oral inhalation part, wherein the plasma aerosol outputted by the plasma treatment module is transported to a respiratory system through at least one of the nasal inhalation part and the oral inhalation part.

The present invention confirms that the plasma aerosol can be applied to treat respiratory-related diseases, such as eliminate sputum/dissolve sputum. For example, according to a preferable embodiment of the present invention, the sputum may be thinned or diluted when the radicals in the plasma water aerosol react with the sputum. Accordingly, the present invention further provides a method for thinning the sputum, which comprises the following steps: delivering an effective amount of a plasma aerosol to a respiratory system of a subject in need. Furthermore, the present invention also provides a use of plasma-treated liquid in manufacture of nebulized inhalant. The plasma-treated liquid serves as an inhalant, the plasma-treated liquid is nebulized into an aerosol form; therefore the inhalant may be delivered to the respiratory system through inhalation and the desired therapeutic effect can be achieved.

Accordingly, the radicals generated by the plasma may only be carried by the aerosol and delivered to the respiratory system to minimize the impact of the radical to other tissues or organs. In addition, since the radicals will self-degrade in the air, there will be no excess accumulation of the radicals.

In the present invention, the inhalation element can be a nasal mask, an oral mask, an oral-nasal mask, a nasal conduit, or an oral conduit. Here, the nasal mask has a nasal inhalation part so that the user may inhale the plasma aerosol into the respiratory system through his/her nose; the oral mask has an oral inhalation part so that the user may inhale the plasma aerosol into the respiratory system through his/her nose; the oral-nasal mask has an oral inhalation part and a nasal inhalation part so that the user may inhale the plasma aerosol into the respiratory system through his/her mouth and nose; the nasal conduit is inserted to the user's nose and has a nasal inhalation part so that the user may inhale the plasma aerosol through his/her nose; the oral conduit is inserted to the user's mouth and has an oral inhalation part so that the user may inhale the plasma aerosol into the respiratory system through his/her mouth.

In the present invention, the plasma generating unit generates plasma using air and oxygen as a reaction gas. A nozzle diameter of the nebulization module is below 20 μm so that the droplet particle size of the aerosol generated by the nebulization module is under 20 μm. Preferably, the droplet particle size of the aerosol generated by the nebulization module is under 10 μm for reaching a better lung deposition amount. More preferably, the droplet particle size of the aerosol generated by the nebulization module is under 5 μm. Accordingly, the efficiency of delivering the aerosol into the respiratory system can be adjusted by the droplet particle size of the aerosol, for example, the droplet particle size of the aerosol may be adjusted between 1-20 μm, preferably between 1-10 μm, and more preferably between 1-5 μm.

### BRIEF DESCRIPTION OF THE DRAWINGS

FIG. 1 is a block diagram of the plasma aerosol inhalation device of one embodiment of the present invention;

FIG. 2 is a flow chart of the method of delivering the plasma aerosol into the respiratory system of one embodiment of the present invention;

FIG. 3 is a block diagram of the plasma aerosol inhalation device of another embodiment of the present invention;

FIG. 4 is a flow chart of the method of delivering the plasma aerosol into the respiratory system of another embodiment of the present invention;

FIG. 5 is the result of the microrheological properties of the artificial sputum (30 mg/ml mucin) in the control group (DI water) and the experimental group (plasma water aerosol); and

FIG. 6 is the result of the microrheological properties of the artificial sputum with different viscosity (30 mg/ml, 60 mg/ml) in the control group (DI water) and the experimental group (plasma water aerosol).

### DETAILED DESCRIPTION OF THE PREFERRED EMBODIMENT

Hereafter, examples will be provided to illustrate the embodiments of the present invention. Advantages and effects of the invention will become more apparent from the disclosure of the present invention. Other various aspects also may be practiced or applied in the invention, and various modifications and variations can be made without departing from the spirit of the invention based on various concepts and applications.

### Embodiment 1

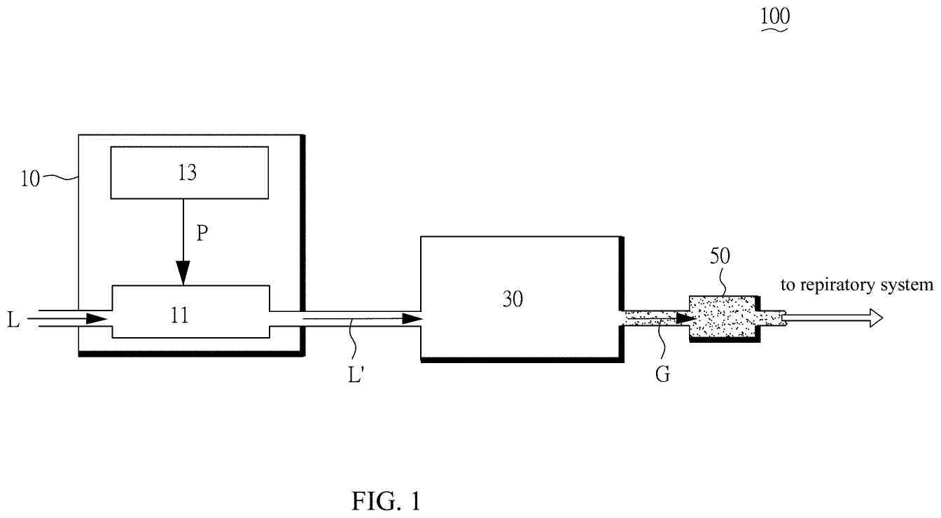

Please refer to FIG. 1, which shows the block chart of the plasma aerosol inhalation device of the present embodiment. As illustrated in FIG. 1, the plasma aerosol inhalation device 100 comprised: a plasma treatment module 10 including a treatment chamber 11 and a plasma generating unit 13, wherein a liquid L was accommodated in the treatment chamber 11 and a plasma P generated by the plasma generating unit 13 was applied to the liquid L in the treatment chamber 11 for forming a plasma-treated liquid L′; a nebulization module 30 interconnected with the treatment chamber 11 of plasma treatment module 10, wherein the plasma-treated liquid L′ exported from the treatment chamber 11 was nebulized into a plasma aerosol G; and an inhalation element 50, which interconnected with the nebulization module, wherein the plasma aerosol G outputted from the nebulization module 30 was delivered to the respiratory system through the inhalation element 50.

In the present embodiment, the liquid L was deionized water (DI water), and the plasma-treated liquid L′ was plasma-treated water. The plasma-treated water was then nebulized into a plasma water aerosol G with 1-10 μm droplet particle size. Here, the plasma generating unit 13 included a quartz tube with 3 mm inner diameter and 5 mm outer diameter, and a platinum electrode with 1 mm diameter and 20 mm length disposed inside the quartz tube. 2 slm (standard liter per minute) of two different low-pressure gas, which were compressed air (21% O₂+79% N₂) and oxygen (99.99% O₂), were inputted into the quartz tube. The platinum electrode is connected with an AC signal, having a 20 kHz frequency and 3˜4 kV voltage, in order to form a low-pressure plasma. The quartz tube is covered with a ceramic tube with 5 mm inner diameter and 8 mm outer diameter for avoiding electric arc.

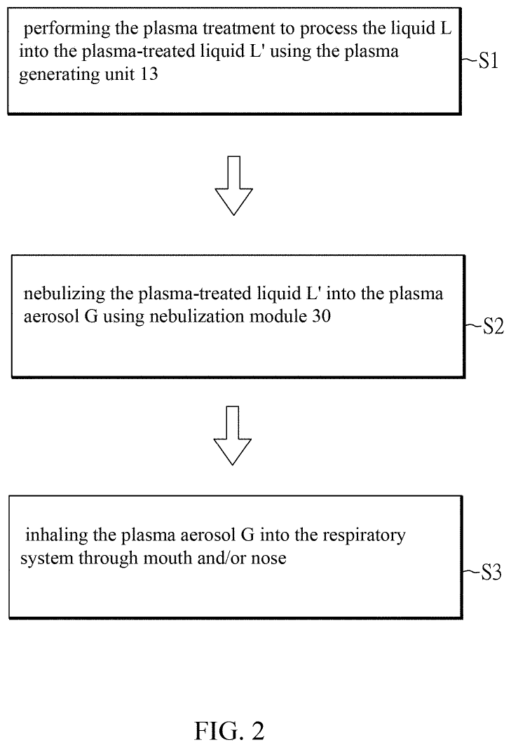

According to the method flow chart shown in FIG. 2, the present embodiment provides a method of delivering a plasma aerosol into a respiratory system. The plasma aerosol may be administered to the respiratory system through the steps in the following paragraph and please refer to the block diagram of the plasma aerosol inhalation device 100 illustrated in FIG. 1 at the same time.

Step S1: performing the plasma treatment to process the liquid L into the plasma-treated liquid L′ using the plasma generating unit 13. Here, the quartz tube is immersed in 100 ml of DI water (liquid L) in 40 mm to perform the plasma treatment. After approximately 10 minutes of plasma treatment, the plasma water (plasma-treated liquid L′) is obtained.

Step S2: nebulizing the plasma-treated liquid L′ into the plasma aerosol G using nebulization module 30. In order to deliver the plasma water (plasma-treated liquid L′) to the respiratory system, the nozzle diameter of the nebulization module 30 was below 10 μm so that the droplet particle size of the plasma water aerosol (plasma aerosol G) was 1˜10 μm.

Step S3: inhaling the plasma aerosol G into the respiratory system through mouth and/or nose. Here, the inhalation element 50 may be used for inhaling the plasma water aerosol (plasma-treated aerosol G) into the respiratory system. The inhalation element 50 may include at least one of a nasal inhalation part and an oral inhalation part. For example, the inhalation element may be a nasal mask, an oral mask, an oral-nasal mask, a nasal conduit, or an oral conduit.

### Embodiment 2

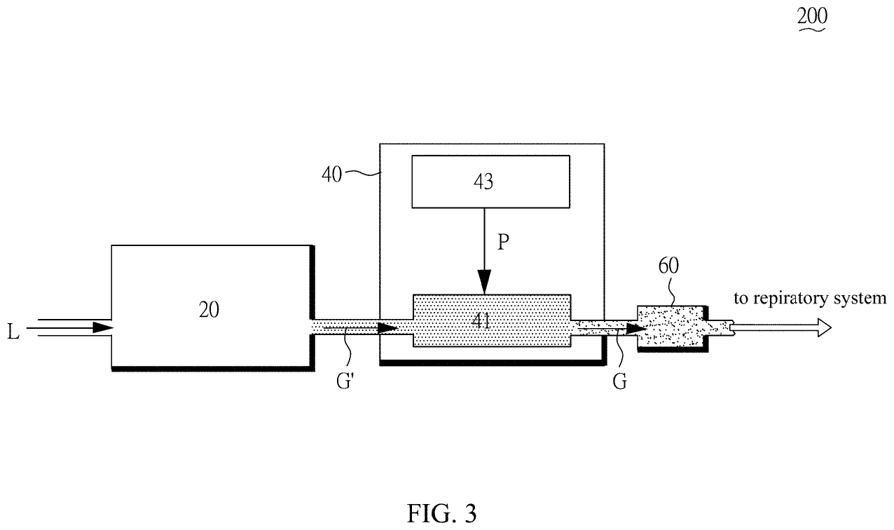

Please refer to FIG. 3, which shows the block diagram of the plasma aerosol inhalation device of another embodiment of the present invention. As illustrated in FIG. 3, the plasma aerosol inhalation device 200 of the present embodiment comprised: a nebulization module 20 nebulizing a liquid L to an aerosol G′; a plasma treatment module 40 including a treatment chamber 41 and a plasma generating unit 43, wherein the treatment chamber 41 was interconnected to the nebulization module for accommodating the aerosol G′ exported by the nebulization module 20, and a plasma P was generated by the plasma generating unit 43 to process the aerosol G′ in the treatment chamber into a plasma aerosol G; and an inhalation element 60 interconnected to the treatment chamber 41 of the nebulization module 40, wherein the plasma aerosol G exported by the treatment chamber 41 was delivered to the plasma aerosol G to the respiratory system through the inhalation element 60.

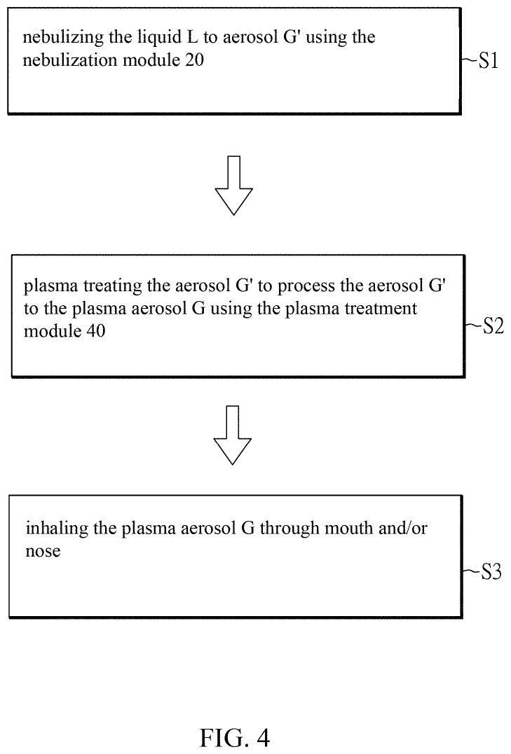

According to the method flow chart illustrated in FIG. 4, the present embodiment provides another method for delivering the plasma aerosol into the respiratory system. The plasma aerosol may be administered to the respiratory system through the steps in the following paragraph and please refer to the block diagram of the plasma aerosol inhalation device 200 illustrated in FIG. 3 at the same time.

Step S1: nebulizing the liquid L to aerosol G′ using the nebulization module 20. The nozzle diameter of the nebulization 20 was below 20 μm so that the droplet particle size of the water aerosol (aerosol G′) was 1˜20 μm.

Step S2: plasma treating the aerosol G′ to process the aerosol G′ to the plasma aerosol G using the plasma treatment module 40, wherein various radicals (ROS/RNS) generated by the plasma were carried by the water aerosol (aerosol G′) after the water aerosol was treated with plasma.

Step S3: inhaling the plasma aerosol G through mouth and/or nose. This step was the same as described in Embodiment 1, and the same description need not be repeated.

### Test Example

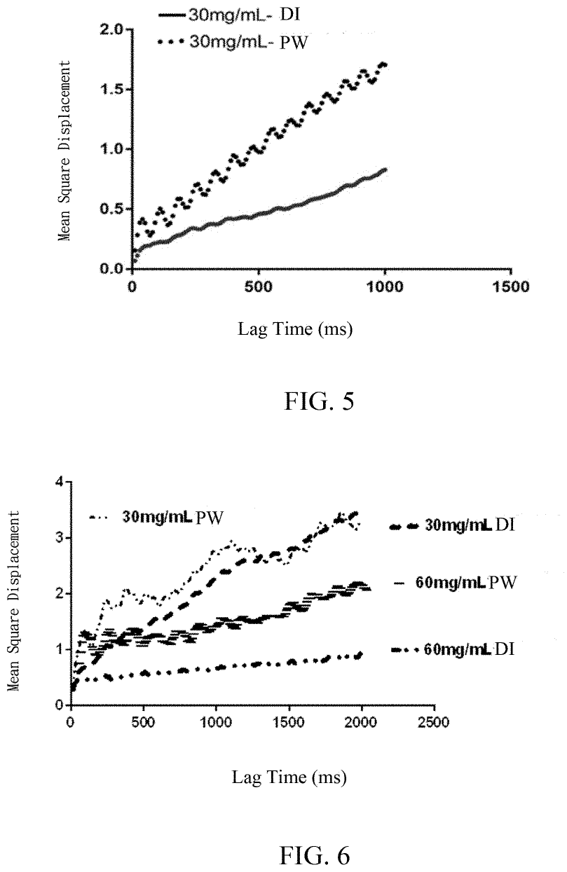

Artificial sputum was prepared by dissolving mucin (10 mg/ml˜60 mg/ml) in saline solution in order to stimulate the sputum of different concentration in the respiratory system under different health conditions. The water aerosol (droplet particle size 1˜20 μm) prepared by the aforementioned methods were added directly to the artificial sputum. The viscous-elastic properties of the sputum were analyzed by multiple-particle tracking. According to the test results shown in FIG. 5 and FIG. 6, the rate of change of the ratio of Mean squared displacement of the particle to lag time of the experimental groups administered with the plasma water aerosol (PW) was significantly higher compared to that of the control group (DI water; DI). This result showed that the plasma water aerosol was able to significantly decrease the viscosity of the sputum.

Accordingly, the present invention verified that the plasma aerosol may be applied to thin the sputum. Therefore, the plasma-treated liquid may serve as an inhalant, the inhalant may be delivered into the respiratory system by nebulizing the plasma-treated liquid to disperse and thin the sputum in practical applications. More specifically, the radicals (ROS/RNS) carried by the plasma water aerosol may react with mucin, DNA molecules, and actin microfilament in the sputum to degrade those biological long-chain polymers in the sputum so that the object of diluting the sputum is achieved.

1. A plasma aerosol inhalation device, comprising:
a plasma treatment module including a treatment chamber and a plasma generating unit, wherein a liquid is accommodated in the treatment chamber and the liquid in the treatment chamber is treated by a plasma generated by the plasma generating unit so as to form a plasma-treated liquid;
a nebulization module interconnecting with the plasma treatment module, wherein the plasma-treated liquid exported from the plasma treatment module is nebulized into a plasma aerosol; and
an inhalation element interconnecting with the nebulization module and having at least one of the nasal inhalation part and an oral inhalation part, wherein the plasma aerosol outputted by the nebulization module is transported to a respiratory system through at least one of the nasal inhalation part and the oral inhalation part.

2. The plasma aerosol inhalation device of claim 1, wherein the liquid is water, the plasma-treated liquid is plasma-treated water, and the plasma aerosol is plasma water aerosol.

3. A plasma aerosol inhalation device, comprising:
a nebulization module nebulizing a liquid into an aerosol;
a plasma treatment module including a treatment chamber and a plasma generating unit, wherein the treatment chamber is interconnected to the nebulization module, wherein the aerosol exported from the nebulization module is accommodated in the treatment chamber and the aerosol in the treatment chamber is treated by a plasma generated by the plasma generating unit so as to form a plasma aerosol; and
an inhalation element interconnected to the plasma treatment module and having at least one of a nasal inhalation part and an oral inhalation part, wherein the plasma aerosol outputted by the plasma treatment module is transported to a respiratory system through at least one of the nasal inhalation part and the oral inhalation part.

4. The plasma aerosol inhalation device of claim 3, wherein the liquid is water, the aerosol is water aerosol, and the plasma aerosol is plasma water aerosol.

5. The plasma aerosol inhalation device of claim 1, wherein the inhalation element is a nasal mask, an oral mask, an oral-nasal mask, a nasal conduit, or an oral conduit.

6. The plasma aerosol inhalation device of claim 1, wherein the plasma generating unit generates plasma using air and oxygen as a reaction gas.

7. The plasma aerosol inhalation device of claim 1, wherein a nozzle diameter of the nebulization module is below 20 μm.

8. A method for thinning the sputum, comprising the following steps: delivering an effective amount of plasma aerosol to a respiratory system of a subject in need using the plasma aerosol inhalation device of claim 1.

9. The method of claim 8, wherein the plasma aerosol is a plasma water aerosol.

10. The method of claim 8, wherein a droplet particle size of the plasma aerosol is under 20 μm.
